(19) United States
(12) Patent Application Publication
(10) Pub. No.: US 2004/0186528 A1
(43) Pub. Date: Sep. 23, 2004

(54) Subcutaneous implantable medical devices with anti-microbial agents for chronic release

(75) Inventors: Richard D. Ries, Stillwater, MN (US); Kenneth E. Cobian, St. Anthony, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/393,121

(22) Filed: Mar. 20, 2003

(51) Int. Cl. A61N 1/375

(52) U.S. Cl. 607/36

(57) ABSTRACT

An anti-microbial component of the IMD that is exposed to body fluids in the pocket is compounded of an anti-microbial metal ion zeolite that elutes metal ions in concentrations exhibiting anti-microbial activity over a substantial period of time of implantation is disclosed. The anti-microbial component is physically attached to the IMD to be retained in close proximity and in a stable location in the subcutaneous pocket. In another embodiment, the anti-microbial component conforms to the shape of the IMD and is attachable to and detachable from the IMD. In another embodiment, the polymeric component includes a connector header of an IPG or a monitor, or a connector sleeve or the sealing rings of a proximal connector assembly of an electrical medical lead coupled with an IPG or monitor that are located in the subcutaneous pocket or in the backing of a subcutaneously implanted cardioversion/defibrillation (C/D) electrode.

### FIELD OF THE INVENTION

The present invention relates generally to implantable medical devices (IMDs), and more particularly to a polymeric member associated with the IMD and compounded from a polymer and an anti-bacterial agent to provide anti-microbial protection during chronic implantation.

### BACKGROUND OF THE INVENTION

At present, a wide variety of IMDs are commercially released or proposed for clinical implantation that include a housing that is implanted subcutaneously and typically include elongated medical electrical leads or drug delivery catheters that extend from the subcutaneous site to other subcutaneous sites or deeper into the body to organs or other implantation sites. Typically, the IMD includes a battery-powered implantable pulse generator (IPG) that is coupled with electrical medical leads, a battery-powered implantable monitor that may or may not be coupled with electrical medical leads, a battery-powered drug pump coupled with a drug delivery catheter, etc. Such IMDs include implantable cardiac pacemakers, cardioverter/defibrillators having pacing capabilities, other electrical stimulators including spinal cord, deep brain, nerve, and muscle stimulators, drug delivery systems, cardiac and other physiologic monitors, cochlear implants, etc.

Typically, the battery-powered component of the IMD is implanted subcutaneously at a surgically prepared site, referred to as a “pocket”, that can be accessed readily when it is necessary to replace the battery-powered component. The surgical preparation and initial and replacement IMD implantations are conducted in a sterile field, and the IMD components are packaged in sterile containers or sterilized prior to introduction into the sterile field. However, despite these precautions, there always is a risk of introduction of microbes into the pocket. Surgeons therefore typically apply disinfectant or antiseptic agents to the skin at the surgical site prior to surgery (e.g., Chlorhexidine, Gluconate, Povidone-Iodine, Isopropyl Alcohol, Ethyl Alcohol), directly to the site before the incision is closed (e.g., gentamicin, vancomycin), and prescribe oral antibiotics for the patient to ingest during recovery (e.g., sefuroxin, gentamicin, rifamycin, vancomycin).

Resident inflammatory cells in the fibrous tissue surrounding the IPG and lead become weakened or “exhausted” over time, such that at the time of IPG replacement, the amount of bacteria that can cause infection in the pocket is reduced by several orders of magnitude. Once the pocket becomes infected, the infection can migrate along the lead sheath to the heart, and such a migrating infection can become intractable and life-threatening, requiring removal of the IPG and lead and drug treatment to cure the infection. Removal of a chronically implanted lead can be difficult and dangerous, and in some cases could require a thoracotomy.

There is a long history of the actual or proposed use of certain elemental metals and metal ions that exhibit anti-microbial behavior in association with a wide variety of products, including IMDs or temporarily implanted devices and instruments, particularly catheters. The metal ions that have been shown to possess antibiotic or anti-microbial activity include silver, gold, platinum, palladium, iridium, antimony, arsenic, selenium, copper, zinc, mercury, tin, lead, and bismuth. Anti-microbial metal ions of silver, gold, copper and zinc, in particular, are considered safe for in vivo use. Anti-microbial silver ions have been found to be particularly useful for in vivo use due to the fact that they are not substantially absorbed into the body. The incorporation of elemental metals into IMDs, particularly silver incorporated into heart valve sewing rings, is proposed in U.S. Pat. No. 6,267,782.

Metallic silver has also been impregnated in the surfaces of medical implants, e.g., catheters, by ion-beam-assisted deposition or implantation as described in U.S. Pat. Nos. 5,474,797 and 5,520,664. The products described in these patents, however, do not exhibit an antibiotic effect for a prolonged period of time because a passivation layer typically forms on the silver metal coating. This layer reduces the release rate of the silver metal from the product, resulting in lower antibiotic effectiveness.

Various compounds have been developed for coating catheters and other devices that release silver ions into body fluids and tissues. As set forth in U.S. Pat. Nos. 6,123,925 and 6,296,863, antibiotic zeolites are well known and have been prepared by replacing all or part of the ion-exchangeable ions in zeolite with ammonium ions and antibiotic metal ions, as described in U.S. Pat. Nos. 4,923,450, 4,938,958, 4,911,898, and 5,100,671. “Zeolite” is a natural or synthetic aluminosilicate having a three dimensional skeletal structure that is represented by the empirical formula: XM_{2/n}O—Al₂O₃—YSiO₂—ZH₂O, wherein M represents an ion-exchangeable ion, generally a monovalent or divalent metal ion, n represents the atomic valency of the (metal) ion, X and Y represent coefficients of metal oxide and silica respectively, and Z represents the number of water of crystallization. Examples of such zeolites include A-type zeolites, X-type zeolites, Y-type zeolites, T-type zeolites, high-silica zeolites, sodalite, mordenite, analcite, clinoptilolite, chabazite and erionite. Such zeolites have been incorporated in antibiotic resins as shown in U.S. Pat. Nos. 4,938,955 and 4,906,464 and polymer articles as shown in U.S. Pat. No. 4,775,585 in concentrations sufficient to effective as an anti-microbial agent. The above-referenced '450 and '671 patents disclose coatings of anti-microbial metal ion zeolites in a polymer, e.g., silicone rubber, on the surface of medical devices, e.g., catheters. In the '925 and '863 patents, particular ones of the above-described antibiotic zeolites are incorporated into coatings applied to porous fabrics used to form implantable vascular grafts and into toothpaste formulations, respectively, in concentrations providing anti-microbial activity.

However, applying coatings of the types described to surfaces of IMDs intended for long-term implantation can be problematic since the coatings can degrade and slough away over time.

### BRIEF SUMMARY OF THE INVENTION

The present invention is directed to providing a simple, effective and long-lasting anti-microbial agent into the subcutaneous implantation pocket that is surgically prepared to receive an IMD of the type described above. In accordance with one aspect of the present invention, an anti-microbial component of the IMD that is exposed to body fluids in the pocket is compounded of an antibiotic zeolite that elutes metal ions in concentrations exhibiting anti-microbial activity over a substantial period of time of implantation. The anti-microbial component is physically attached to the IMD to be retained in close proximity and in a stable location in the subcutaneous pocket.

In one embodiment, the anti-microbial component conforms to the shape of the IMD and is attachable to and detachable from the IMD. The anti-microbial component includes a polymeric pad or boot that fits around at least a portion of an outer housing of the IMD, wherein the IMD may include an ICD IPG, a pacemaker IPG, a neurostimulator IPG, a muscle stimulator IPG, a monitor, a drug pump, or a subcutaneous electrode or components thereof that implanted subcutaneously. The surgeon can exercise the option of using or not using the anti-microbial component in any particular instance whether based on medical or aesthetic considerations. Moreover, it is not necessary for manufacturers to commit to manufacturing and clinical buyers to stock redundant models of expensive IMDs, one model with the anti-microbial polymeric component and one without the anti-microbial polymeric component.

In another embodiment, the polymeric component includes a connector header of an IPG or a monitor or the sealing rings of a proximal connector assembly of an electrical medical lead coupled with an IPG or monitor that are located in the subcutaneous pocket or in the backing of a subcutaneously implanted cardioversion/defibrillation (C/D) electrode.

Polymeric boots have been proven over long-term clinical use to not degrade significantly in the body despite the fact that they are relatively thin. Therefore, it is expected that metal (e.g., silver) silver ions of the anti-microbial agent dispersed through the thin wall of the anti-microbial pad or boot component or other component will be beneficially released over time.

This summary of the invention has been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIG. 1 is a schematic view of an implantable medical device, according to the present invention, implanted subcutaneously in a patient's thoracic region, having a silicone rubber boot compounded with metal ion zeolite fitted over the device

FIG. 2 is a plan view of the silicone rubber boot compounded of metal ion zeolite of FIG. 1;

FIG. 3 is a side-cross-section view of the boot taken along lines 3-3 of FIG. 2;

FIG. 4 is a top view of the boot of FIG. 2;

FIG. 5 is a schematic view of an implantable medical device according to the present invention including, implanted subcutaneously in a patient's thoracic region, having a silicone rubber boot compounded with metal ion zeolite fitted over the device and having a further silicone rubber boot compounded with metal ion zeolite fitted over or attached to the non-conducting side of the device;

FIG. 6 is a schematic view of an implantable medical device according to the present invention included two modules implanted subcutaneously across the patient's thorax and tethered together, each module having a silicone rubber boot compounded with metal ion zeolite fitted over the device;

FIG. 7 is a schematic view of an implantable medical device according to the present invention implanted subcutaneously in a patient's thoracic region having a silicone rubber boot compounded with metal ion zeolite fitted over the device;

FIG. 8 is a schematic view of an implantable medical device according to the present invention implanted subcutaneously in a patient's thoracic region having a silicone rubber boot compounded with metal ion zeolite fitted over the device;

FIG. 9 is a schematic view of an implantable medical device according to the present invention implanted subcutaneously in a patient's thoracic region having a silicone rubber boot compounded with metal ion zeolite fitted over the device;

FIG. 10 is a schematic view of an implantable medical device according to the present invention implanted subcutaneously in a patient's thoracic region having a silicone rubber boot compounded with metal ion zeolite fitted over the device;

FIG. 11 is a schematic view of an implantable medical device according to the present invention implanted subcutaneously in a patient's thoracic region having a silicone rubber boot compounded with metal ion zeolite fitted over the device;

FIG. 12 is a schematic partial view of an exemplary implantable medical device according to the present invention depicting a connector header in partial cross-section and an exemplary lead connector assembly adapted to be fitted into a connector bore, wherein selected ones or all of polymeric components of the connector header and/or the lead connector assembly are compounded with metal ion zeolite in accordance with an embodiment of the present invention; and

FIG. 13 is a perspective view of a subcutaneously implantable C/D electrode wherein selected ones or all of the polymeric components of the C/D electrode are compounded with metal ion zeolite in accordance with an embodiment of the present invention.

### DETAILED DESCRIPTION OF THE INVENTION

In the following detailed description, references are made to illustrative embodiments of methods and apparatus for carrying out the invention. It is understood that other embodiments can be utilized without departing from the scope of the invention.

In the preferred embodiments, an inorganic anti-microbial agent is incorporated into a polymeric component of or a detachable boot that can be optionally fitted against or over the housing of an IMD that is subcutaneously implanted, particularly a monitor, a drug pump, an IPG and subcutaneously implanted electrodes or sensors. The inorganic anti-microbial agent is preferably the antibiotic silver ion zeolite the type designated HealthShield™, which is sold by AgION™ Technologies, Inc., the assignee of the above-referenced '925 and '863 patents.

This material is basically an anti-microbial zeolite of the types described above having a metal having one or the whole of the metal substituted by at least one kind of an ion exchangeable metal selected from the group consisting of Ag, Cu and Zn. A typical particle size for the agent is between 0.8 and 10 microns. The particles are dispersed in silicone rubber in the quantity of between 0.5 and 20% by weight, more preferably between 0.5 and 15% by weight and most preferably between 0.5 and 10% by weight. The silicone rubber-particle mixture is molded into a desired shape employing conventional medical grade silicone rubber molding techniques. In accordance with the invention, other inorganic anti-microbial metal ions, e.g., gold, platinum, palladium, iridium, antimony, arsenic, selenium, copper, zinc, mercury, tin, lead, bismuth, cadmium, chromium and thallium ions can be employed instead of silver.

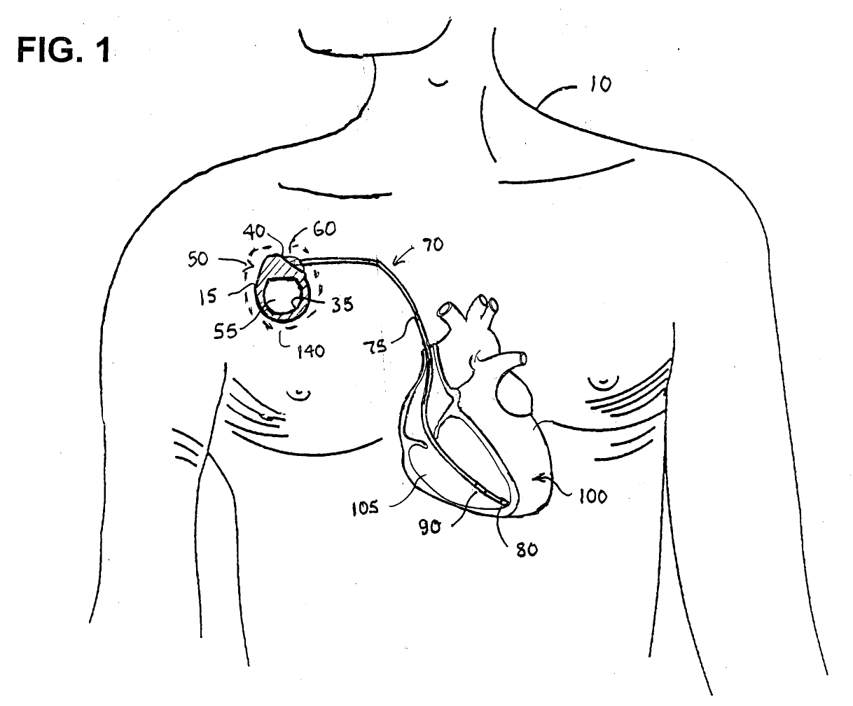

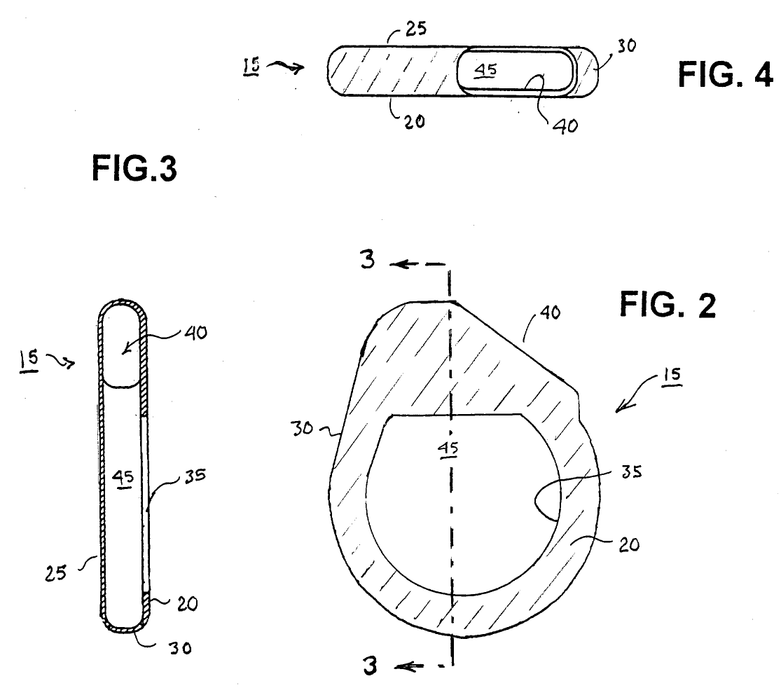

A first embodiment of a detachable, elastic, boot 15 that is compounded of silicone rubber and the preferred anti-microbial metal ion zeolite and molded in a shape to be fitted over an IPG or monitor 50 implanted in patient 10 is depicted in FIGS. 1-4. The boot 15 has first and second major boot sides 20 and 25 joined by a mutual boot edge 30 defining a boot cavity 45. A side opening 35 through major boot side 20 and an edge opening 40 through a segment of boot edge 30 are provided.

The boot 15 is fitted over the housing 55 and connector block 60 of the exemplary IPG or monitor and inserted into a subcutaneous pocket 140 at a distance from the heart 100 as shown in FIG. 1. The fitted boot 15 provides the anti-microbial protection in the subcutaneous implantation pocket 140 while leaving at least a portion of the housing 55 of IPG/monitor 50 exposed through side opening 35. The IPG 50 is depicted in FIG. 1 as a ventricular pacemaker IPG or hemodynamic monitor that is coupled to a cardiac lead 70 extending from a connection with connector block 60 into the heart 100 through a conventional transvenous route. The cardiac lead comprises an active or cathodal pace/sense electrode 80 at the distal end of lead body 75 and optionally comprises a pressure transducer 90 proximal to pace/sense electrode 80 both disposed in this instance in the right ventricle 105 of heart 100. The housing 55 of IPG 50 is hermetically sealed and formed of a conductive metal that is electrically connected to pacing and/or sensing circuitry within housing 55 to function as an indifferent or anodal pace/sense electrode 85 that is exposed by side opening 35.

The housing 55 and connector block 60 of IPG/monitor 50 can take any shape known in the art, and that shape dictates the shape and dimensions of the boot 15. The specifications and operating modes and other characteristics of the pacemaker IPG and the cardiac lead(s) coupled therewith can correspond to any of those known in the art. The monitor can correspond to the Medtronic® CHRONICLE® IHM that is coupled through a cardiac lead of the type described in commonly assigned U.S. Pat. No. 5,564,434 having capacitive blood pressure and temperature sensors as well as at least one EGM sense electrode.

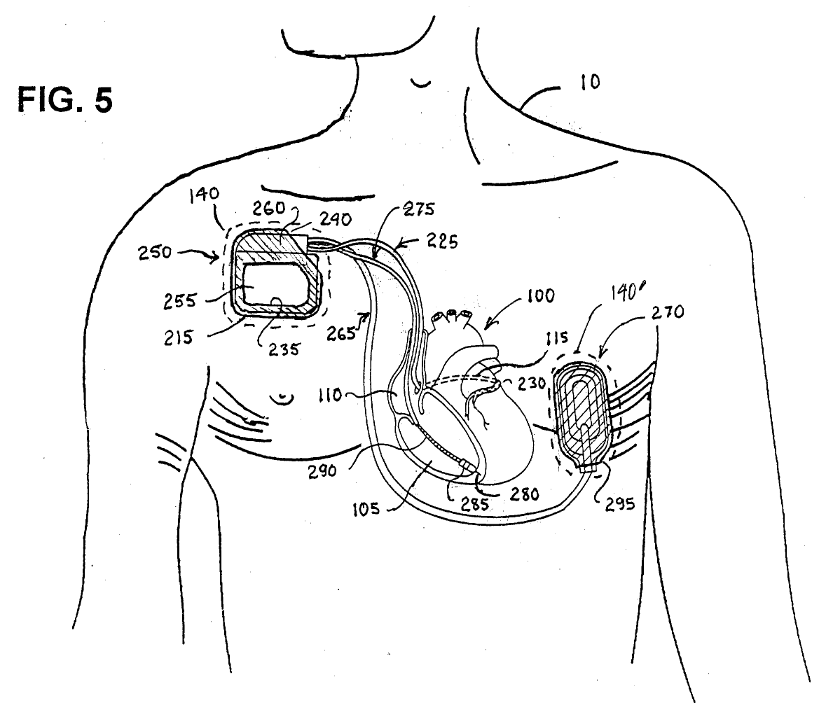

The IPG/monitor 50 is slipped through the side opening 35 and the connector block 60 is oriented to be exposed through the edge opening 40. It will also be understood that the side opening 35 is necessary to expose the housing 55 for use as a remote indifferent pacing and/or sensing electrode in either of a unipolar pacemaker IPG/monitor 50 or in a bipolar pacemaker IPG/monitor also having the capability of monitoring the far field EGM. The boot 15 having such a side opening 35 can still be efficaciously used over a typical bipolar pacemaker IPG/monitor not having such a far field sensing capability. These features of the boot 15 are applicable to the remaining boot embodiments illustrated in FIGS. 5-10.

A second embodiment of a detachable, elastic, boot 215 that is compounded of silicone rubber and the preferred anti-microbial metal ion zeolite and molded in a shape to be fitted over a rectilinear ICD IPG 250 implanted in patient 10 is depicted in FIG. 5. The boot 215 is also formed of first and second major boot sides joined by a mutual boot edge defining a side opening 235 through major boot side and an edge opening 240 through a segment of the boot edge.

The boot 215 is fitted over the housing 255 and connector block 260 of the exemplary ICD IPG 250 and inserted into a subcutaneous pocket 140 at a distance from the heart 100 as shown in FIG. 5. The fitted boot 215 provides the anti-microbial protection in the subcutaneous implantation pocket 140 while leaving at least a portion of the housing 255 of ICD IPG 250 exposed through side opening 235. The exposed portion of the housing 255 may be employed as one C/D electrode.

The ICD IPG 250 depicted in FIG. 5 is coupled to an exemplary set of C/D leads extending to pace/sense electrodes and C/D electrodes. It will be understood that not all of the depicted C/D leads and that other combinations of C/D leads can be connected to the ICD IPG 250. In this particular instance, a right ventricular (RV) C/D lead 275 extends from a connection with connector block 260 into the right ventricle 105 of the heart 100 through a conventional transvenous route. The RV C/D lead 275 comprises active or cathodal pace/sense electrode and fixation helix 280 at the distal end of the lead body, a more proximally located, ring-shaped, indifferent or anodal pace/sense electrode 285, and an elongated C/D electrode 290. A coronary sinus (CS) C/D lead 225 extends from a connection with connector block 260 to an elongated C/D electrode 230 disposed in the coronary sinus or great vein 115 of the heart 100 through a conventional transvenous route.

A further C/D lead 265 extends subcutaneously from a connection with connector block 260 to a rectilinear, pad-shaped, C/D electrode 270 disposed in a further subcutaneous pocket 140′ selected by the surgeon to optimally apply C/D shock therapies between selected pairs of the C/D electrodes 230, 255, 270, and 290. Typically the rectilinear C/D electrode 270 is formed of a flexible silicone rubber or polyurethane pad supporting a C/D electrode surface or array on one major side disposed toward heart 100 and a non-conductive side disposed toward the skin. A further detachable, elastic, boot 295 that is compounded of silicone rubber and the preferred anti-microbial metal ion zeolite and molded in a shape to be fitted over the non-conductive major side of the rectilinear C/D electrode 270 is shown in FIG. 5. The boot 295 can be affixed by sutures or other means to the silicone rubber or polyurethane pad to ensure that it does not move or detach from the non-conductive side within the pocket 140′.

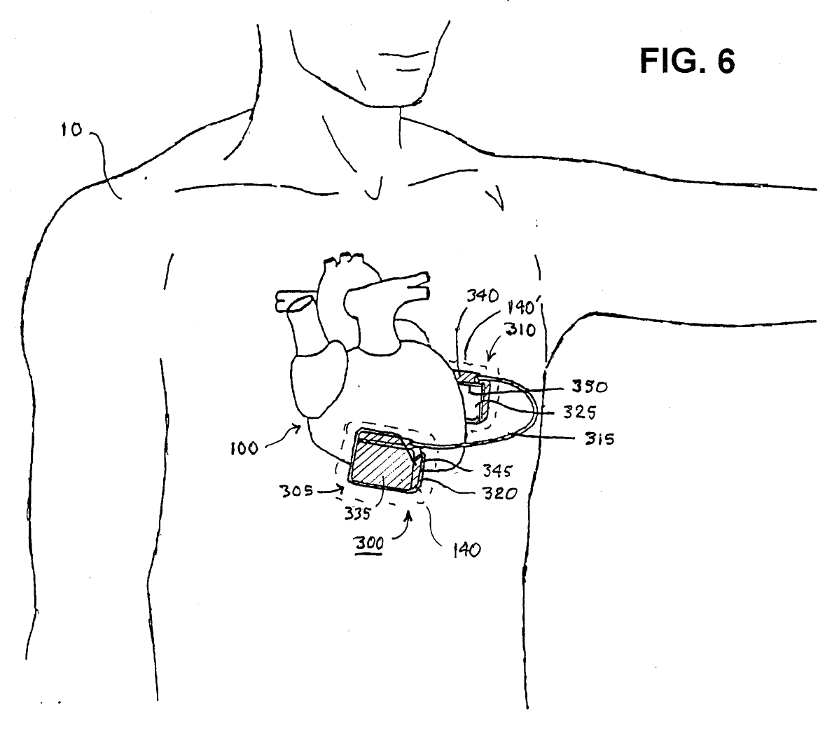

More recently, it has been proposed that all components of an ICD be implanted subcutaneously distributed between two or more C/D electrode bearing modules implanted in subcutaneous pockets 140, 140′ around the thorax to deliver C/D shock therapies between them and through the heart. Such ICDs are disclosed in U.S. Pat. Nos. 5,255,692, 5,314,451, and 5,342,407 and in U.S. Patent Application Publication Nos. 2002/0042634 and 2002/0035377. Such an arrangement is depicted in FIG. 6 wherein the ICD 300 comprises first and second schematically depicted, hermetically sealed ICD IPG modules 305 and 310 tethered together by a cable 315. First and second C/D electrodes 320 and 325 are supported on one side of the ICD IPG modules 305 and 310, respectively, that are intended to be implanted in the subcutaneous pockets 140, 140′ facing the heart 100 and one another.

The hermetically sealed ICD IPG module 305 encloses the electronic sensing, pacing, and C/D circuitry, including the relatively bulky high voltage capacitors that are charged and discharged to deliver C/D shocks, as well as a low voltage battery employed for powering the circuitry and the delivered pacing pulses. The second hermetically sealed ICD IPG module 310 encloses a relatively bulky high power C/D battery as well as a switch to enable selective connection with the high voltage capacitor charging circuitry within the first ICD IPG module 305 in the manner described in the above-referenced '451 patent. The cable 315 encases conductors distributing power from the battery and exchanging signals and commands between circuitry in the first and second ICD IPG modules 305 and 310.

First and second detachable, elastic, boots 335 and 340 that are each compounded of silicone rubber and the preferred anti-microbial metal ion zeolite and molded in a shape to be fitted over the respective first and second ICD IPG modules 305 and 310 implanted in patient 10 are also depicted in FIG. 6. The boots 335 and 340 have openings 345 and 350 in the major sides thereof that expose the first and second respective C/D electrodes 320 and 325.

The first and second hermetically sealed ICD IPG modules 305 and 310 bearing the first and second detachable, elastic, boots 335 and 340 are preferably implanted subcutaneously in posterior and anterior positions through a single skin incision intermediate the illustrated posterior and anterior positions. Tunneling tools would be employed to displace the tissue and advance the first and second hermetically sealed housings to the depicted sites or other selected sites around the thorax. Tissue adhesive may be employed to secure the first and second hermetically sealed ICD IPG modules 305 and 310 bearing the first and second detachable, elastic, boots 335 and 340 at the sites and prevent migration. Alternatively, the sites may be exposed through minimal surgical exposures, and the first and second hermetically sealed ICD IPG modules 305 and 310 bearing the first and second detachable, elastic, boots 335 and 340 can be sutured at the sites through the boots 335 and 340 to prevent migration.

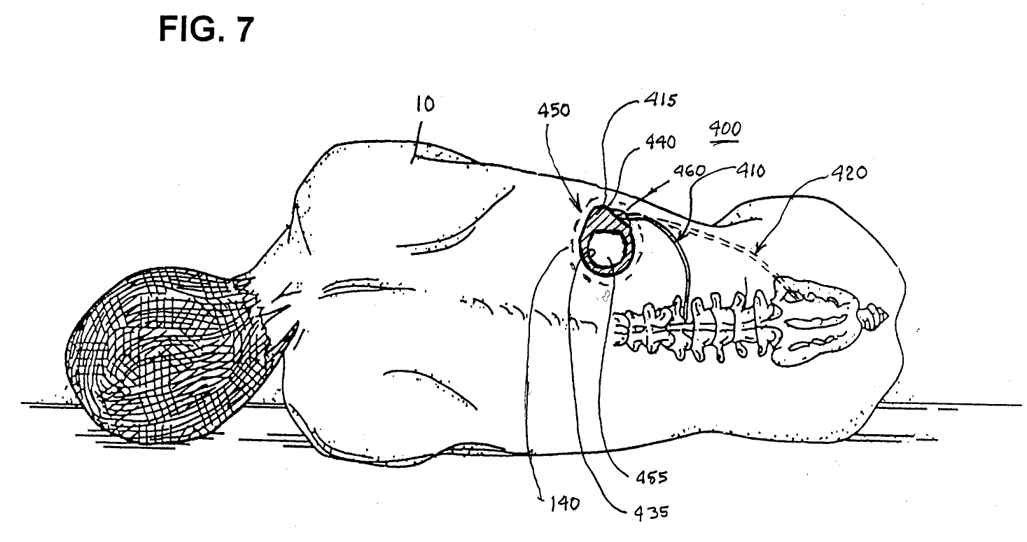

Therapeutic administration of pain suppressing electrical stimulation into the intraspinal space, that is to either the epidural space or to the intrathecal space, is also known in the art as illustrated in FIG. 7. Three meningeal sheaths that are continuous with those which encapsulate the brain within the enclosure by the vertebral canal for the spinal cord by the bones of the vertebrae surround the spinal cord. The outermost of these three meningeal sheaths is the dura matter, a dense, fibrous membrane which anteriorally is separated from the periosteum of the vertebral by the epidural space. Posterior to the dura matter is the subdural space. The subdural space surrounds the second of the three meningeal sheaths, the arachnoid membrane, which surround the spinal cord. The arachnoid membrane is separated from the third meningeal sheath, the pia mater, by the subarachnoid or intrathecal space. The subarachnoid space is filled with CSF. Underlying the pia mater is the spinal cord. Thus the progression proceeding inwards or in posterior manner from the vertebra is the epidural space, dura mater, subdural space, arachnoid membrane, intrathecal space, pia matter and spinal cord.

An exemplary spinal cord stimulation (SCS) system 400 comprising a neurostimulator SCS IPG 450, an SCS lead 410, and a detachable, elastic, boot 415 that is each compounded of silicone rubber and the preferred anti-microbial metal ion zeolite and molded in a shape to be fitted over the housing and connector of the neurostimulator IPG 450 is depicted implanted in patient 10 in FIG. 7. The neurostimulator IPG 450 may comprise the Medtronic® Itrel® 3, Synergy™ or Synergy Versitrel™ neurostimulator, and the SCS lead 410 may comprise the Medtronic® Pisces Z Quad lead.

Therapeutic administration of stimulation of the sacral nerves to control bladder function or treat sexual dysfunction is also alternatively illustrated in FIG. 7 by the sacral nerve stimulation lead 420 depicted in dotted lines extending from the neurostimulator IPG 450 and detachable, elastic, boot 415 into a foramen of the sacrum. In this case, the neurostimulator IPG 450 may comprise the Medtronic® InterStim® Neurostimulator Model 3023. In one embodiment, a sacral nerve stimulation lead 420 bearing one or a plurality of distal stimulation electrodes are percutaneously implanted through the dorsum and the sacral foramen of the sacral segment S3 for purposes of selectively stimulating the S3 sacral nerve. The distal electrode(s) is positioned using a hollow spinal needle through a foramen (a singular foramina) in the sacrum. The electrode is secured by suturing the lead body in place, and the lead body is tunneled subcutaneously to the implant site of the neurostimulator IPG 450 within the boot 415.

The detachable, elastic, boot 415 corresponds to the detachable, elastic, boot 15 described above with respect to FIGS. 1-4. It will be understood that the actual shape of such commercially available neurostimulator IPGs may differ from the exemplary shape of neurostimulator IPG 450 shown in FIG. 7, and that boot 415 is molded to conform to the actual shape. Again, the boot 415 has a major side opening 435 exposing the housing 455 of the IPG 450 that can function as an indifferent stimulation electrode in conjunction with a stimulation electrode or electrodes along the distal end segment of the SCS lead 410 disposed within the intraspinal space and obscured from view. The boot 415 also has an edge opening 440 enabling access to the connector block 460.

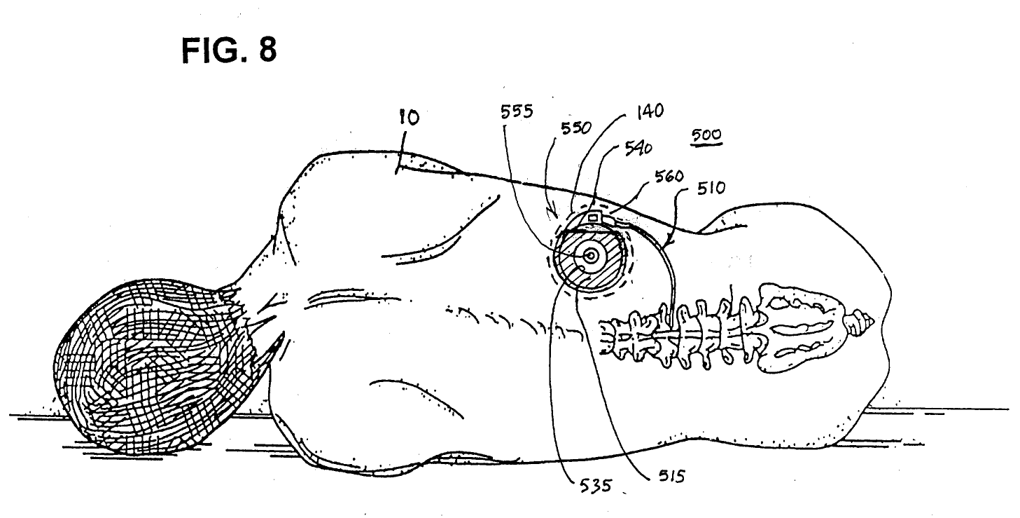

Therapeutic administration of pain suppression or therapeutic drugs into the intraspinal space as also known in the prior art is illustrated in FIG. 8. Administration of a drug directly to the intrathecal space can be by either spinal tap injection or by catheterization. Intrathecal drug administration can avoid the inactivation of some drugs when taken orally as well and the systemic effects of oral or intravenous administration. Additionally, intrathecal administration permits use of an effective dose that is only a fraction of the effective dose required by oral or parenteral administration. Furthermore, the intrathecal space is generally wide enough to accommodate a small catheter, thereby enabling chronic drug delivery systems. Thus, it is known to treat spasticity by intrathecal administration of baclofen. Additionally, it is known to combine intrathecal administration of baclofen with intramuscular injections of botulinum toxin for the adjunct effect of intramuscular botulinum for reduced muscle spasticity. Furthermore, it is known to treat pain by intraspinal administration of the opioids morphine and fentanyl. A drug pump is required because the antinociceptive or antispasmodic drugs in current use have a short duration of activity and must therefore be frequently re-administered, which re-administration is not practically carried out by daily spinal tap injections. The drug pump is surgically placed under the skin of the patient's abdomen. One end of a catheter is connected to the pump, and the other end of the catheter is threaded into a CSF filled subarachnoid or intrathecal space in the patient's spinal cord. The implanted drug pump can be programmed for continuous or intermittent infusion of the drug through the intrathecally located catheter.

Thus a fully implantable intrathecal drug delivery system 500, e.g., the Medtronic® SynchroMed® EL Infusion System, comprising a programmable SynchroMed® drug pump 550 and a drug delivery catheter 510, is depicted in FIG. 8. A detachable, elastic, boot 515 that is compounded of silicone rubber and the preferred anti-microbial metal ion zeolite and molded in a shape to be fitted over the housing and connector of the drug pump 550 is depicted implanted in patient 10 in FIG. 7. Again, the boot 515 has a major side opening 535 in this case exposing a drug fill port 555 for percutaneously refilling a drug chamber within the drug pump 550 in a manner well known in the art. The boot 515 also has an edge opening 540 enabling access to the connector block 560 that the drug delivery catheter 510 is attached to. The drug pump 550 and boot 515 encasing the drug pump 550 are implanted just under the skin of the abdomen in a prepared subcutaneous pocket 140 so that the drug fill port is oriented outward to enable access to the drug fill port 555.

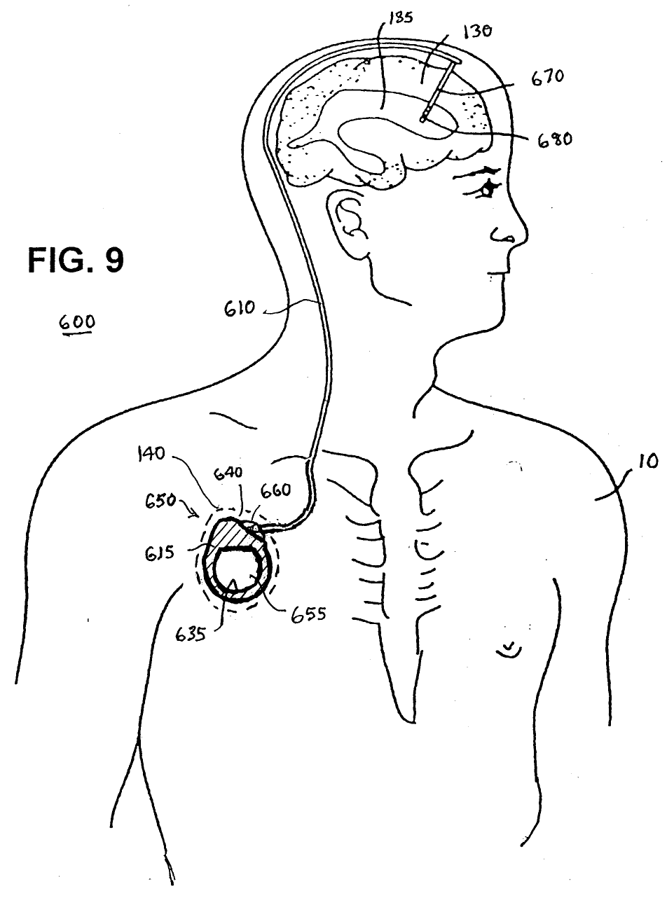

Turning to FIG. 9, it schematically illustrates the delivery of Medtronic® Activa® Tremor Control Therapy or Parkinson's Control Therapy to a patient 10 for controlling essential tremors and those associated with Parkinson's disease. The Activa® Therapy is delivered by an deep brain stimulator similar to a cardiac pacemaker, that uses mild electrical stimulation delivered by electrodes implanted in the brain to block the brain signals that cause tremor.

The Activa Tremor Control System stimulates targeted cells in the thalamus—the brain's message relay center—via electrodes that are surgically implanted in the brain and connected to a neurostimulator IPG implanted near the collarbone. In the treatment of Parkinson's tremors, the electrodes are located at the subthalamic nucleus (STN) or globus pallidus interna (GPI) that control movement and muscle function. A lead with tiny electrodes is surgically implanted at these sites in the brain and connected by an extension that lies under the skin to a neurostimulator IPG implanted near the collarbone. The electrical stimulation can be non-invasively adjusted to meet each patient's needs.

The implanted components of the Activa® System 600 depicted in FIG. 9 include the Medtronic® Itrel® II Model 7424 neurostimulator IPG 650, a DBS™ lead 670 and an extension 610 that connects the lead 670 to the neurostimulator IPG 650. The lead 670 is implanted using a stereotactic headframe designed to keep the head stationary and help guide the surgeon in the placement of the lead 670 into the brain 130 to dispose the electrodes 680 at the desired site 135. The brain 130 and the placement of the lead 670 is imaged using CT (computed tomography) or MRI (magnetic resonance imaging) equipment. The Model 3387 DBS™ lead, with a plurality of widely spaced electrodes, and the Model 3389 DBS™ lead, with a plurality of narrowly spaced electrodes, provide physician options for precise placement and stimulation selectivity. Other components of the Activa® System 60 include a neurostimulator control magnet, neurological test stimulator, physician programmer, lead frame kits, and MemoryMod® software cartridge.

A detachable, elastic, boot 615 that is compounded of silicone rubber and the preferred anti-microbial metal ion zeolite and molded in a shape to be fitted over the housing and connector block of the neurostimulator IPG 650 is depicted implanted in patient 10 in FIG. 9. Again, the boot 615 has a major side opening 635 and an edge opening 640 enabling access to the connector block 660 that the lead extension 610 is attached to. The neurostimulator IPG 650 and boot 615 encasing the neurostimulator IPG 650d are implanted just under the skin of the upper thorax in a prepared subcutaneous pocket 140. The exposed surface of the bipolar neurostimulator housing 655 can be employed as a stimulation electrode in this instance.

An implantable infusion pump (IIP) comprising an implantable drug pump and catheter is disclosed in commonly assigned U.S. Pat. Nos. 5,643,207 and 5,782,798 for dispensing pancreatic polypeptide blockers and other drugs that decrease sensations of hunger and increase satiety into particular sites in the brain through a distal catheter segment that is implanted through the skull and extends to the specific sites. The delivery of other appetite influencing drugs directly into the brain for increasing appetite to treat anorexia is also proposed in the '207 patent. The drug that is dispensed from the infusion pump coupled to the catheter through the catheter lumen and into the brain is expected to induce or increase the feeling of satiety to treat obesity by reducing caloric intake or to increase feelings of hunger to treat anorexia by increasing caloric intake. The system of the '798 patent can also be employed to apply electrical stimulation to the brain through catheter borne electrodes and conductors to increase feelings of satiety to treat obesity or to decrease feelings of satiety to treat anorexia presumably either with of without delivery of the identified drugs.

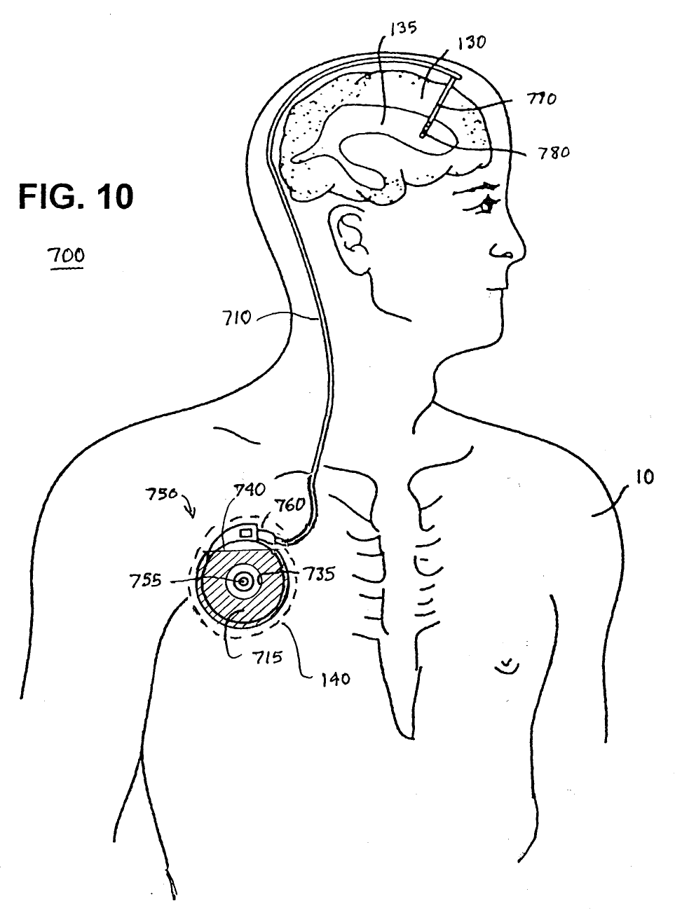

Such an implantable deep brain drug delivery system 700 is depicted in FIG. 10 comprising an implantable drug pump 750 and catheter 710 for dispensing pancreatic polypeptide blockers and other drugs that decrease sensations of hunger and increase satiety through catheter ports 780 into a particular site 135 in the brain 130 through a distal catheter segment 770 that is implanted through the skull and extends to the specific site 135. The implantable drug pump 750 can comprise a programmable SynchroMed® drug pump 750. A detachable, elastic, boot 715 that is compounded of silicone rubber and the preferred anti-microbial metal ion zeolite and molded in a shape to be fitted over the housing and connector of the drug pump 750 is depicted implanted in patient 10 in FIG. 10. Again, the boot 715 has a major side opening 735 in this case exposing a drug fill port 755 for percutaneously refilling a drug chamber within the drug pump 750 in a manner well known in the art. The boot 715 also has an edge opening 740 enabling access to the connector block 760 that the drug delivery catheter 710 is attached to. The drug pump 750 and boot 715 encasing the drug pump 750 are implanted just under the skin of the thorax in a prepared subcutaneous pocket 140 so that the drug fill port is oriented outward to enable access to the drug fill port 755.

An implantable EGM monitor for recording the cardiac electrogram from electrodes remote from the heart is disclosed in commonly assigned U.S. Pat. No. 5,331,966 and PCT publication WO 98/02209 and is embodied in the Medtronic® REVEAL® Model 9526 Insertable Loop Recorder having spaced housing EGM electrodes employed with a Model 6191 patient activator and a Model 9790 programmer. Such implantable monitors when implanted in patients suffering from cardiac arrhythmias or heart failure accumulate date and time stamped data that can be of use in determining the condition of the heart over an extended period of time and while the patient is engaged in daily activities. A wide variety of other IMDs have been proposed to monitor many other physiologic conditions as set forth in U.S. Pat. No. 6,221,011.

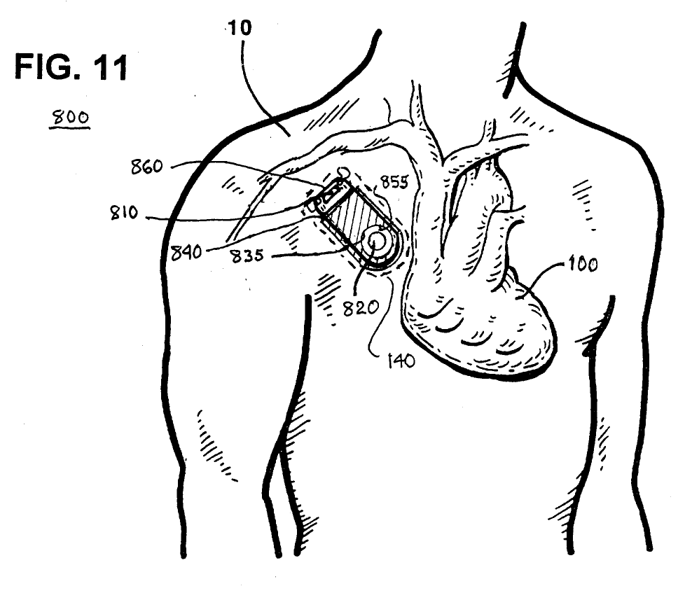

Therefore, a REVEAL® Insertable Loop Recorder 850 is depicted in FIG. 11 implanted in a subcutaneous pocket 140 in the thorax of patient 10. The Insertable Loop Recorder 850 comprises a hermetically sealed housing 855 enclosing the monitoring circuitry, battery, telemetry antenna, and other components and a header 860 that supports a sense electrode 810 coupled to the a sense amplifier via a feedthrough extending through the housing 855 and has a pair of suture holes extending through it. An electrically uninsulated portion of the housing 855 that is coupled with the sense amplifier provides a second sense electrode 820. A detachable, elastic, boot 815 that is compounded of silicone rubber and the preferred anti-microbial metal ion zeolite and molded in a shape to be fitted over at least the housing 855. Again, the boot 815 has a major side opening 835 exposing the sense electrode 820 and an edge opening 840 enabling insertion of the housing 855 into the boot 815. The boot 815 may be shaped to extend over at least the portions of the header 860 having the suture holes to enable using the same sutures to secure the boot to the Insertable Loop Recorder 850 and the Insertable Loop Recorder 850 to subcutaneous tissue.

Thus, a variety of subcutaneously implanted IMDs have been described having a variety of uses and shapes that are implanted in subcutaneous pockets 140, 140′ and over which a detachable anti-microbial component characterized as a pad or boot that fits around at least a portion of an outer housing of the IMD is placed. The subcutaneous site is advantageously protected from microbial growth and infections of the types described above by inclusion of the anti-microbial polymeric component that is exposed to body fluids in the pockets 140, 140′ that is compounded of an antibiotic zeolite that elutes silver ions in concentrations exhibiting anti-microbial activity over a substantial period of time of implantation. In these embodiments depicted in FIGS. 1-11, the anti-microbial component is physically attached to the IMD by fitting it over the IMD. It will be understood that the anti-microbial component can be molded to conform to the shape of any IMD adapted to be implanted subcutaneously that is presently available or may become available in the future, e.g., gastric stimulators and drug pumps, insulin delivery drug pumps, and other body organ, muscle or nerve stimulators and drug delivery devices that are specifically identified herein.

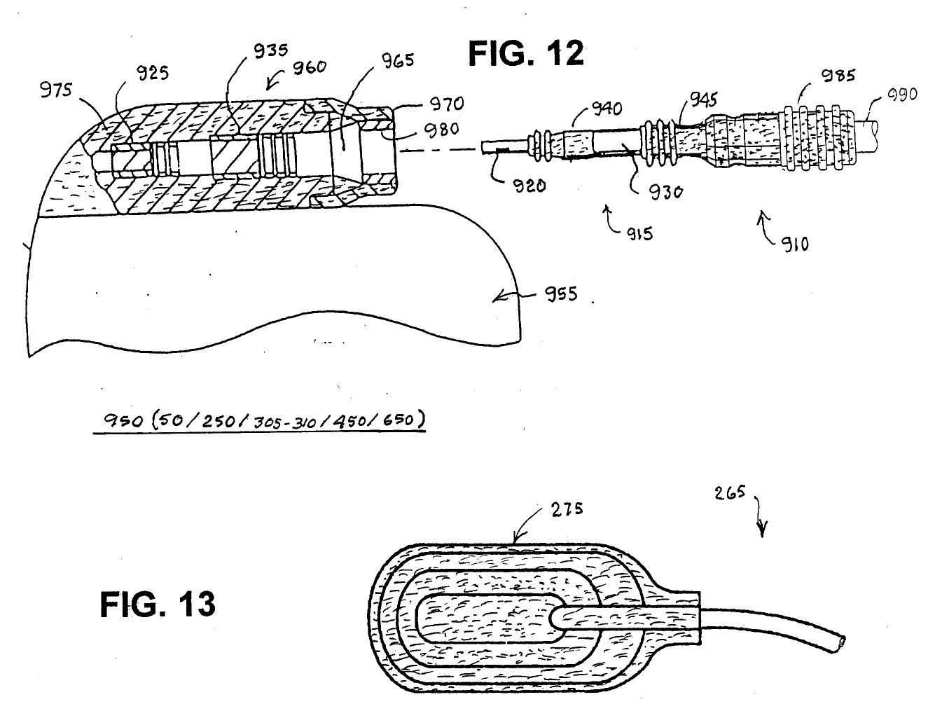

In another preferred embodiment, the anti-microbial component comprises a permanently attached portion of any of the above-identified IMDs that are implanted into the prepared subcutaneous pocket 140. For example, a schematic partial view of an exemplary IPG/monitor 950 depicting the connector header 960 in partial cross-section and an exemplary lead connector assembly 915 of an electrical medical lead 910 adapted to be fitted into a connector bore 965, is depicted in FIG. 12. A bipolar lead 910 is depicted having a connector assembly 915 of conventional bipolar design comprising a connector pin 920 and a connector ring 930 adapted to fit a pin receptacle contact 925 and a ring receptacle contact of schematically depicted connector header 960. Elastic polymeric sealing rings 940 and 945 are located adjacent to the connector pin 920 and connector ring 930. A distal portion 985 of the lead connector assembly 915 coupled to the elongated lead body 990 is disposed outside the connector bore 965 when the more proximal portion of the lead connector assembly 915 is fully inserted within the connector bore 965. Elastic bands 970 and 980 encircle the connector bore opening and a suture can be applied to tighten them against the elastic portion of the connector assembly between the sealing rings 945 and the distal portion 955. The particular configurations of the connector elements 925 and 935, the feedthroughs and wire connections, and any setscrews or other fasteners that are encased within the molded polymeric header body 975 for making secure electrical connections can take any of the known configurations and are not important to the practice of the present invention and are not depicted. The depicted IPG/monitor 950 is exemplary of any of the IPG/monitors and components thereof 50, 250, 305-310, 450, and 650, although the number of connector elements of the lead connector assembly and the connector header and their specific configurations may vary widely.

Selected ones or all of the polymeric components of the IPG connector header 975 and/or the lead connector assembly 915 are compounded with metal ion zeolite as indicated by the hatching in FIG. 12 in accordance with a further embodiment of the invention. Usually, the lead connector assembly 915 is separately formed and attached to the lead body 990 in manufacture, so it is convenient to mold the polymeric lead connector assembly parts from silicone rubber or polyurethane compounded with the metal ion zeolite. The anti-microbial silver ions can thereby be eluted from the connector header body 975 and/or from the elastic band 970 and or from the lead connector portion 985 that is disposed outside the connector bore 965. The anti-microbial silver ions can also be eluted from the sealing rings 940 and 945 if they become wet with body fluids over chronic implantation to inhibit any microbial activity within the connector bore/connector assembly interface.

FIG. 13 is a perspective view of a subcutaneously implantable C/D electrode, e.g., C/D electrode 275 wherein selected ones or all of the polymeric components of the C/D electrode 275 are compounded with metal ion zeolite in accordance with a further embodiment of the invention. In particular, all or portions of the silicone rubber or polyurethane pad 220 can be molded with the metal ion zeolite as indicated by the hatching in FIG. 13. Again, the silicone rubber or polyurethane pad 220 is separately formed and attached to the lead body of C/D lead 265 in manufacture, so it is convenient to mold the polymeric pad as a single part or as multiple parts, depending on the design, from silicone rubber or polyurethane compounded with the metal ion zeolite.

Similarly, the polymeric header 860 of the implantable monitor 800, for example, the subcutaneously tunneled cable 315, for example, between subcutaneously implanted IMD components, and the polymeric component of the catheter connectors 560 and 760 with the implantable drug pumps 500 and 700, for example, can be molded from polymers compounded with metal ion zeolite.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. A method of providing anti-microbial protection in a subcutaneous pocket in which an implantable medical device (IMD) having a predetermined IMD shape is implanted comprising:
providing an anti-microbial component that conforms to the IMD shape and is selectively attachable to and detachable from the IMD;
attaching the anti-microbial component to the IMD; and
implanting the IMD and the anti-microbial component within the subcutaneous pocket.

2. The method of claim 1, wherein the providing step further comprises: compounding a biocompatible polymer with a metal ion zeolite in sufficient concentration to provide anti-microbial activity by elution of metal ions into body fluids; and
molding the compounded biocompatible polymer with the metal ion zeolite into the anti-microbial component into a thin-walled, elastic boot adapted to fit over at least a portion of the IMD that is wettable by body fluids to enable elution of the metal ions into body fluid.

3. The method of claim 2, wherein the metal ions are selected from the group consisting of silver, gold, platinum, palladium, iridium, antimony, arsenic, selenium, copper, zinc, mercury, tin, lead, bismuth, cadmium, chromium and thallium ions.

4. The method of claim 3, wherein the IMD comprises one of the group consisting of an electrode, an implantable pulse generator, and a drug pump.

5. The method of claim 2, wherein the molding step further comprises molding the elastic boot with an internal boot cavity shaped to receive at least a portion of the IMD and at least one opening facilitating insertion of the IMD into the boot cavity in the attaching step and removal of IMD from the boot cavity.

6. The method of claim 5, wherein the IMD supports an electrode surface, and the attaching step comprises aligning the at least one opening in relation to the electrode surface to expose the electrode surface.

7. The method of claim 1, wherein the providing step further comprises molding the anti-microbial component as a thin-walled, elastic boot adapted to fit over at least a portion of the IMD.

8. The method of claim 7, wherein the molding step further comprises molding the elastic boot with an internal boot cavity shaped to receive at least a portion of the IMD and at least one opening facilitating insertion of the IMD into the boot cavity in the attaching step and removal of IMD from the boot cavity.

9. The method of claim 8, wherein the IMD supports an electrode surface, and the attaching step comprises aligning the at least one opening in relation to the electrode surface to expose the electrode surface.

10. The method of claim 1, wherein the providing step further comprises molding the anti-microbial component as a thin-walled, elastic pad adapted to be attached to at least a portion of the IMD.

11. The method of claim 1, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.

12. A method of providing anti-microbial protection in a subcutaneous pocket in which an implantable medical device (IMD) having a biocompatible polymeric component is implanted comprising:
forming at least a portion of the polymeric component of the IMD with an anti-microbial IMD component; and
implanting the IMD with the anti-microbial IMD component within the subcutaneous pocket.

13. The method of claim 12, wherein the forming step further comprises: compounding a biocompatible polymer with a metal ion zeolite in sufficient concentration to provide anti-microbial activity by elution of metal ions into body fluids; and
molding the compounded biocompatible polymer with the metal ion zeolite into the anti-microbial IMD component.

14. The method of claim 13, wherein the metal ions are selected from the group consisting of silver, gold, platinum, palladium, iridium, antimony, arsenic, selenium, copper, zinc, mercury, tin, lead, bismuth, cadmium, chromium and thallium ions.

15. The method of claim 13, wherein the IMD comprises the combination of one of the group consisting of an implantable pulse generator (IPG) and a monitor, each having a connector header, combined with an electrical medical lead having a connector assembly that is received in a connector bore of the connector assembly, and the molding step comprises molding at least a portion of the connector header of the compounded biocompatible polymer and metal ion zeolite.

16. The method of claim 15, wherein the IPG comprises one of the group consisting of a group consisting of sacral nerve stimulator IPG, a spinal cord stimulator IPG, a cardiac pacing IPG, a deep brain stimulator IPG, and an implantable cardioverter/defibrillator IPG formed of a single IPG module or plural IPG modules.

17. The method of claim 13, wherein the IMD comprises the combination of one of the group consisting of an implantable pulse generator (IPG) and a monitor, each having a connector header, combined with an electrical medical lead having a connector assembly that is received in a connector bore of the connector assembly, and the molding step comprises molding at least a portion of the connector assembly of the compounded biocompatible polymer and metal ion zeolite.

18. The method of claim 17, wherein the IPG comprises one of the group consisting of a sacral nerve stimulator IPG, a spinal cord stimulator IPG, a deep brain stimulator IPG, and an implantable cardioverter/defibrillator IPG formed of a single IPG module or plural IPG modules.

19. The method of claim 13, wherein the IMD comprises a subcutaneously implantable electrode comprising a conductive electrode surface supported on a polymeric backing, and the molding step comprises molding at least a portion of the polymeric backing of the compounded biocompatible polymer and metal ion zeolite.

20. The method of claim 13, wherein the IMD comprises an implantable monitor having a polymeric header, and the molding step comprises molding at least a portion of the header of the compounded biocompatible polymer and metal ion zeolite.

21. The method of claim 13, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.

22. Apparatus for providing anti-microbial protection in a subcutaneous pocket in which an implantable medical device (IMD) having a predetermined IMD shape is implanted comprising:
an anti-microbial component shaped to conform to the shape of the IMD; and means for attaching the anti-microbial component to the IMD.

23. The apparatus of claim 22, wherein the anti-microbial component is compounded of a biocompatible polymer with a metal ion zeolite in sufficient concentration to provide anti-microbial activity by elution of metal ions into body fluids.

24. The apparatus of claim 23, wherein the metal ions are selected from the group consisting of silver, gold, platinum, palladium, iridium, antimony, arsenic, selenium, copper, zinc, mercury, tin, lead, bismuth, cadmium, chromium and thallium ions.

25. The apparatus of claim 23, wherein the IMD comprises one of the group consisting of an electrode, an implantable pulse generator, and a drug pump.

26. The apparatus of claim 23, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.

27. The apparatus of claim 22, wherein the anti-microbial component is compounded of a biocompatible polymer with a metal ion zeolite in sufficient concentration to provide anti-microbial activity by elution of metal ions into body fluids, and the anti-microbial component is molded into a thin-walled, elastic boot adapted to fit over at least a portion of the IMD that is wettable by body fluids to enable elution of the metal ions into body fluid.

28. The apparatus of claim 27, wherein the metal ions are selected from the group consisting of silver, gold, platinum, palladium, iridium, antimony, arsenic, selenium, copper, zinc, mercury, tin, lead, bismuth, cadmium, chromium and thallium ions.

29. The apparatus of claim 27, wherein the IMD comprises one of the group consisting of an electrode, an implantable pulse generator, and a drug pump.

30. The apparatus of claim 27, wherein the elastic boot is molded with an internal boot cavity shaped to receive at least a portion of the IMD and at least one opening facilitating insertion of the IMD into or removal of the IMD from the boot cavity.

31. The apparatus of claim 27, wherein the IMD supports an electrode surface, and the elastic boot is molded with an internal boot cavity shaped to receive at least a portion of the IMD and at least one opening adapted to be aligned with the electrode surface to expose the electrode surface.

32. The apparatus of claim 22, wherein the anti-microbial component is compounded of a biocompatible polymer with an anti-microbial agent in sufficient concentration to provide anti-microbial activity by elution of anti-microbial agent into body fluids, and the anti-microbial component is molded into a thin-walled, elastic boot adapted to fit over at least a portion of the IMD that is wettable by body fluids to enable elution of the metal ions into body fluid.

33. The apparatus of claim 32, wherein the elastic boot is molded with an internal boot cavity shaped to receive at least a portion of the IMD and at least one opening facilitating insertion of the IMD into or removal of the IMD from the boot cavity.

34. The apparatus of claim 32, wherein the IMD supports an electrode surface, and the elastic boot is molded with an internal boot cavity shaped to receive at least a portion of the IMD and at least one opening adapted to be aligned with the electrode surface to expose the electrode surface.

35. The apparatus of claim 22, wherein the anti-microbial component is compounded of a biocompatible polymer with an anti-microbial agent in sufficient concentration to provide anti-microbial activity by elution of anti-microbial agent into body fluids, and the anti-microbial component is molded into a thin-walled, elastic pad adapted to fit against and attached to at least a side of the IMD that is wettable by body fluids to enable elution of the metal ions into body fluid.

36. The apparatus of claim 22, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.

37. A apparatus for providing anti-microbial protection in a subcutaneous pocket in which an implantable medical device (IMD) is implanted comprising:
an IMD housing formed at least in part of a biocompatible polymeric component; and
an anti-microbial agent incorporated into the biocompatible polymeric component to provide an anti-microbial IMD component providing anti-microbial activity by elution of anti-microbial agent into body fluids.

38. The apparatus of claim 37, wherein the anti-microbial IMD component is compounded of a biocompatible polymer with a metal ion zeolite in sufficient concentration to provide anti-microbial activity by elution of metal ions into body fluids and molded into a component shape.

39. The apparatus of claim 38, wherein the metal ions are selected from the group consisting of silver, gold, platinum, palladium, iridium, antimony, arsenic, selenium, copper, zinc, mercury, tin, lead, bismuth, cadmium, chromium and thallium ions.

40. The apparatus of claim 38, wherein the IMD comprises the combination of one of the group consisting of an implantable pulse generator (IPG) and a monitor, each having a connector header, combined with an electrical medical lead having a connector assembly that is received in a connector bore of the connector assembly, and the anti-microbial IMD component comprises at least a portion of the connector header.

41. The apparatus of claim 40, wherein the IPG comprises one of the group consisting of a sacral nerve stimulator IPG, a spinal cord stimulator IPG, a deep brain stimulator IPG, a cardiac pacing IPG, and an implantable cardioverter/defibrillator IPG formed of a single IPG module or plural IPG modules.

42. The apparatus of claim 38, wherein the IMD comprises the combination of one of the group consisting of an implantable pulse generator (IPG) and a monitor, each having a connector header, combined with an electrical medical lead having a lead connector assembly that is received in a connector bore of the connector assembly, and the anti-microbial IMD component comprises at least a portion of the lead connector assembly.

43. The apparatus of claim 42, wherein the IPG comprises one of the group consisting of a sacral nerve stimulator IPG, a spinal cord stimulator IPG, a deep brain stimulator IPG, a cardiac pacing IPG, and an implantable cardioverter/defibrillator IPG formed of a single IPG module or plural IPG modules.

44. The apparatus of claim 38, wherein the IMD comprises a subcutaneously implantable electrode comprising a conductive electrode surface supported on a polymeric backing, and the anti-microbial IMD component comprises at least a portion of the polymeric backing.

45. The apparatus of claim 38, wherein the IMD comprises an implantable monitor having a polymeric header, and the and the anti-microbial IMD component comprises at least a portion of the header.

46. The apparatus of claim 38, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.

47. The apparatus of claim 37, wherein the IMD comprises the combination of one of the group consisting of an implantable pulse generator (IPG) and a monitor, each having a connector header, combined with an electrical medical lead having a connector assembly that is received in a connector bore of the connector assembly, and the anti-microbial IMD component comprises at least a portion of the connector header.

48. The apparatus of claim 47, wherein the IPG comprises one of the group consisting of a sacral nerve stimulator IPG, a spinal cord stimulator IPG, a deep brain stimulator IPG, a cardiac pacing IPG, and an implantable cardioverter/defibrillator IPG formed of a single IPG module or plural IPG modules.

49. The apparatus of claim 48, wherein the IMD comprises the combination of one of the group consisting of an implantable pulse generator (IPG) and a monitor, each having a connector header, combined with an electrical medical lead having a lead connector assembly that is received in a connector bore of the connector assembly, and the anti-microbial IMD component comprises at least a portion of the lead connector assembly.

50. The apparatus of claim 49, wherein the IPG comprises one of the group consisting of a sacral nerve stimulator IPG, a spinal cord stimulator IPG, a deep brain stimulator IPG, a cardiac pacing IPG, and an implantable cardioverter/defibrillator IPG formed of a single IPG module or plural IPG modules.

51. The apparatus of claim 48, wherein the IMD comprises a subcutaneously implantable electrode comprising a conductive electrode surface supported on a polymeric backing, and the anti-microbial IMD component comprises at least a portion of the polymeric backing.

52. The apparatus of claim 48, wherein the IMD comprises an implantable monitor having a polymeric header, and the and the anti-microbial IMD component comprises at least a portion of the header.

53. The apparatus of claim 48, wherein the IMD comprises one of the group consisting of a sacral nerve stimulator implantable pulse generator (IPG), a spinal cord stimulator IPG, a deep brain stimulator IPG, a spinal cord drug pump, a deep brain drug pump, a cardiac pacing IPG, an implantable cardioverter defibrillator IPG, a cardiac hemodynamic monitor, a cardiac monitor, and a cardioversion/defibrillation electrode.
